(19) United States
(12) Patent Application Publication
(10) Pub. No.: US 2018/0144474 A1
(43) Pub. Date: May 24, 2018

(54) THERAPY PLAN DEVICE

(71) Applicant: MITSUBISHI ELECTRIC CORPORATION, Chiyoda-ku, Tokyo (JP)

(72) Inventors: Naoaki MATSUMURA, Tokyo (JP); Hideki FUJI, Tokyo (JP)

(73) Assignee: MITSUBISHI ELECTRIC CORPORATION, Chiyoda-ku, Tokyo (JP)

(21) Appl. No.: 15/577,867

(22) PCT Filed: Jul. 24, 2015
(86) PCT No.: PCT/JP2015/071075

§ 371 Date: Nov. 29, 2017

(51) Int. Cl. G06T 7/00; G06T 1/00; G16H 50/70

(52) U.S. Cl. CPC G06T 7/0014; G06K 9/6201; G16H 50/70; G06T 1/00

(57) ABSTRACT

Similarities and volume ratios are calculated by a diseased-site contour data calculation unit, in such a manner that diseased-site contour data in data of a patient to be treated is compared with respective diseased-site contour data in a therapy plan database, using each of the centers in respective patient-body contour data, as a reference; and then similar cases are searched by an existing therapy-plan data search unit, based on the calculated similarities and the volume ratios, so that the case(s) with a high similarity is utilized as a similar case(s) for preparing a new therapy plan.

### TECHNICAL FIELD

The present invention relates to a therapy plan device in which, from among existing therapy plan data, therapy plan data of a treated case similar to a case of a patient to be treated is searched.

### BACKGROUND ART

In conventional therapy plan devices, when, from among therapy plan data used in the past therapies, therapy plan data similar to that for a patient to be treated is searched, diagnostic information (document-based information), an arbitrarily-set image feature amount and planning data for the patient to be treated are used as search keys. For example, in Patent Document 1, it is disclosed that similarities between a CT image of an object case (a CT image thereof transformed to be positionally matched to CT images of the past treated cases) and the CT images of the past treated cases, are subjected to searching using the image feature amount (that can be arbitrarily set in consideration of a geometric situation of the object case, such as position coordinates of the diseased site) as a search key, so that the treated case with a high similarity is used as a similar case for preparing a therapy plan.

### CITATION LIST

### Patent Document

### Patent Document 1: Japanese Patent Application Laid-open No. 2013-198652 (Paragraph 0024, FIG. 2)

### SUMMARY OF THE INVENTION

### Problems to be Solved by the Invention

However, according to Patent Document 1, because the image feature amount used as a search key is to be arbitrarily set, it is required to prepare the image feature amount after considering what should be defined as that amount. As described so far, according to the conventional therapy plan devices, the diagnostic information (document-based information) and the planning data for the patient to be treated are required to be set in advance for searching, and data of the arbitrarily-set image feature amount has to be prepared for searching. Thus, there is a problem that the time to be taken for advance preparation is prolonged.

The present invention has been made to solve the problem as described above, and an object thereof is to provide a therapy plan device that can easily perform similarity searching with the existing therapy plan data in a short time without requiring advance preparation for that searching.

### Means for Solving the Problems

A therapy plan device of the invention is characterized by comprising: data of a patient to be treated which includes first diseased-site contour data and first body contour data that are read out from image data of the patient to be treated; a therapy plan database which includes respective second diseased-site contour data and respective second body contour data that are read out from respective image data of patients in treated cases; a diseased-site contour data calculation unit for calculating similarities and volume ratios between the first diseased-site contour data and the respective second diseased-site contour data by comparing them using an origin point as a reference point; and an existing therapy-plan data search unit for searching a similar case(s), based on the similarities and the volume ratios;

said origin point being an intersection point between an X-axis and a Y-axis in a cross-section with respect to a body-axis direction, of each of the patient to be treated and the patients in the treated cases, in which the X-axis corresponds to a line that passes a midpoint of a line segment connecting a frontmost-end point and a rearmost-end point in a front-rear direction in each of the first body contour data and the respective second body contour data, and that is perpendicular to the front-rear direction; and the Y-axis corresponds to a line in the front-rear direction that passes a midpoint of a line segment connecting a rightmost-end point and a leftmost end point in a lateral direction in said each of the first body contour data and the respective second body contour data, and

said origin point also being an intersection point between a Z-axis in a longitudinal section along the Y-axis with respect to each said body-axis direction and a sectional plane, in which the Z-axis corresponds to a line perpendicular to the X-axis and the Y-axis that passes the intersection point between the X-axis and the Y-axis, and the sectional plane passes a midpoint of a line segment connecting both-end points having a maximum width in the body-axis direction in said each of the first diseased-site contour data and the respective second diseased-site contour data.

### Effect of the Invention

According to the invention, the similarities and the volume ratios are calculated by the diseased-site contour data calculation unit in such a manner that the diseased-site contour data in the data of a new patient to be treated is compared with the respective diseased-site contour data in the therapy plan database, using the centers of the respective body contour data in the data of the patient to be treated and in each of the treated cases, each as a reference; and then a similar case(s) is searched by the existing therapy-plan data search unit, based on the calculated similarities and volume ratios. This makes it possible to easily perform similarity searching from the existing therapy plan data in a short time without requiring advance preparation for that similarity searching with the existing therapy plan data, and a case(s) with a high similarity can be utilized as a similar case(s) for preparing a new therapy t plan.

### BRIEF DESCRIPTION OF THE DRAWINGS

FIG. 1 is a block diagram showing a configuration of a therapy plan device according to Embodiment 1 of the invention.

FIG. 2A and FIG. 2B are sectional views for illustrating a diseased-site contour data comparing method by the therapy plan device according to Embodiment 1 of the invention.

FIG. 3A and FIG. 3B are enlarged sectional views for illustrating the diseased-site contour data comparing method by the therapy plan device according to Embodiment 1 of the invention.

FIG. 4 is a flowchart for illustrating an existing therapy-plan data searching method by the therapy plan device according to Embodiment 1 of the invention.

### MODES FOR CARRYING OUT THE INVENTION

### Embodiment 1

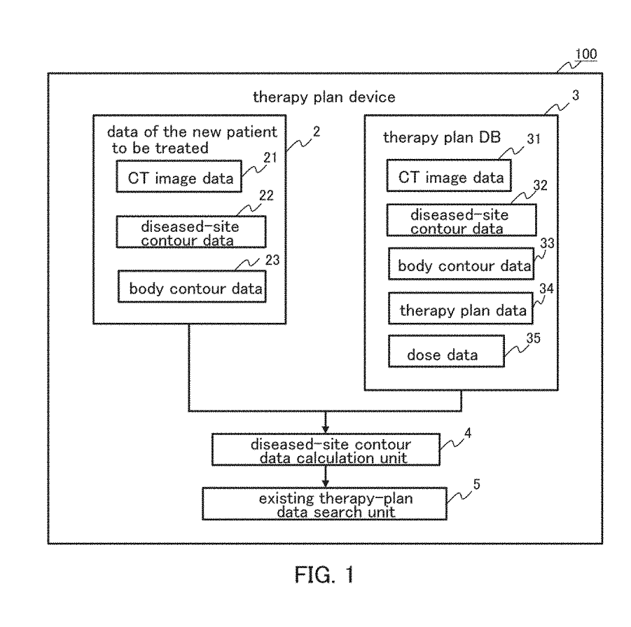

FIG. 1 is a block diagram showing a configuration of a therapy plan device 100 according to Embodiment 1 of the invention. As shown in FIG. 1, the therapy plan device 100 is configured with: data of a patient to be treated 2; a therapy plan database (DB: Data Base) 3; a diseased-site contour data calculation unit 4; and an existing therapy-plan data search unit 5.

The data of a patient to be treated 2 includes: CT (Computed Tomography) image data 21 of a new patient to be treated; diseased-site contour data 22 thereof that is first diseased-site contour data; and body contour data 23 thereof that is first diseased-site contour data; and is stored in a storage unit such as an HDD (Hard Disk Drive) or the like.

The therapy plan database 3 includes: respective CT image data 31 of patients in existing treated cases; respective diseased-site contour data 32 thereof that is respective second diseased-site contour data; respective body contour data 33 thereof that is respective second body contour data; and, as therapy plan information, respective therapy plan data 34 and respective dose data 35; which are stored in a storage unit such as an HDD or the like, on a unit organ basis, for example.

The diseased-site contour data calculation unit 4 compares the diseased-site contour data 22 in the data of a patient to be treated 2 for the new patient to be treated, with the respective diseased-site contour data 32 in the therapy plan database 3 based on the existing treated cases, to thereby calculate similarities between them. Further, it compares the diseased-site contour data 22 in the data of a patient to be treated 2, with the respective diseased-site contour data 32 in the therapy plan database 3, to thereby calculate volume ratios between them.

From the therapy plan database 3, the existing therapy-plan data search unit 5 extracts each treated case, as a similar treated case, which is provided with a high matching rate as the similarity calculated by the diseased-site contour data calculation unit 4 and with a similar volume in terms of the volume ratio calculated by the diseased-site contour data calculation unit 4, and then displays that treated case. The therapy plan data 34, the dose data 35 and the like in the extracted similar treated case can be utilized as therapy plan data for the new patient to be treated.

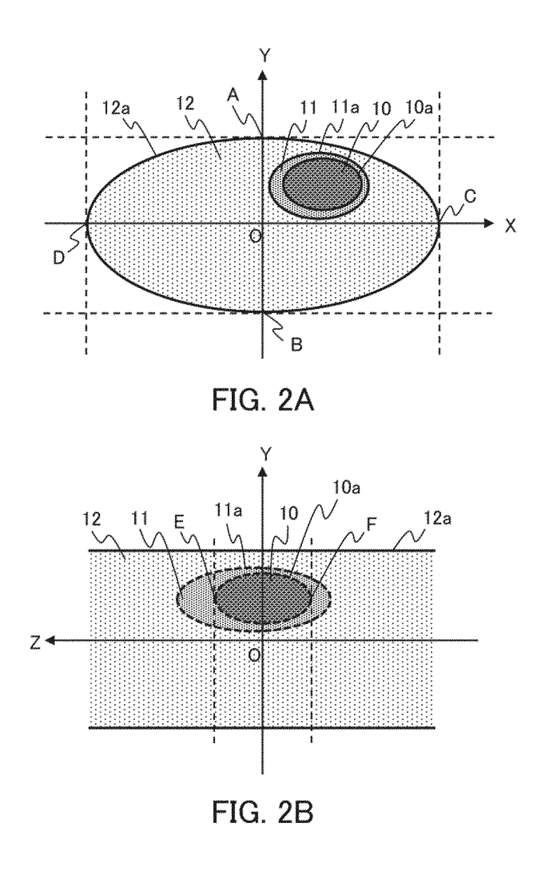

Next, a position to be provided as a reference in comparing the diseased-site contour data 22 with each of the respective diseased-site contour data 32, will be described using FIG. 2A and FIG. 2B. In Embodiment 1 of the invention, FIG. 2A shows a cross-section with respect to a body-axis direction of a patient 12, and FIG. 2B shows a longitudinal section in the body-axis direction.

As shown in FIG. 2A, in the cross-section with respect to the body-axis direction of the patient 12, such a line is defined as an X-axis that passes a midpoint of a line segment connecting a front-end touch point A and a rear-end touch point B in a front-rear direction in the body contour data 12a of the patient 12, and that is perpendicular to the front-rear direction; and such a line in the front-rear direction is defined as a Y-axis that passes a midpoint of a line segment connecting a right-end touch point C and a left-end touch point D in a lateral direction in the body contour data 12a of the patient 12.

Then, as shown in FIG. 2B, in the Y-axis longitudinal section in the body-axis direction, such a line perpendicular to the X-axis and the Y-axis is defined as a Z-axis that passes an intersection point between the X-axis and the Y-axis. As an origin point, such an intersection point is defined that is between a sectional plane and the Z-axis, said sectional plane passing a midpoint of a line segment connecting a touch point E and a touch point F at the both ends having a laterally maximum width in the Z-axis direction in the diseased-site contour data 10a of the patient 12.

The diseased-site contour data 22 and the body contour data 23 in the data of a patient to be treated 2, and the respective diseased-site data 32 and the respective body contour data 33 in the therapy plan database 3, are obtained by image processing of the CT image data 21 in the data of a patient to be treated 2 and the respective CT image data 3 in the therapy plan database 3, respectively. Using the origin point as a reference, the diseased-site contour data 22 in the data of a patient to be treated 2 is compared with the respective diseased-site contour data 32 in the therapy plan database 3, to thereby calculate the similarities and the volume ratios.

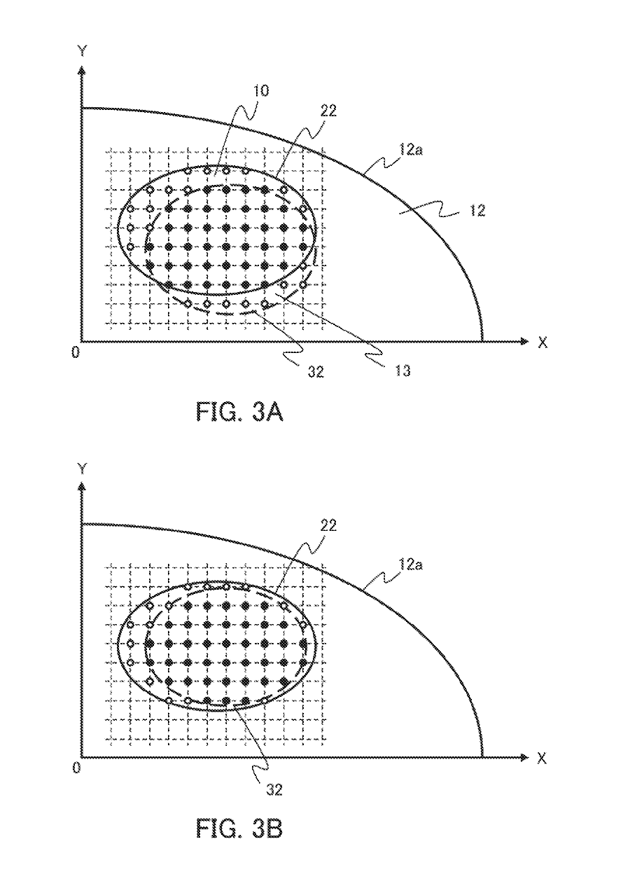

Next, methods for calculating the similarities and the volume ratios will be described. FIG. 3A and FIG. 3B show examples of how to compare the diseased-site contour data 22 with each of the respective diseased-site contour data 32, in a section along the X and Y axes, in which FIG. 3A shows a situation where the diseased-site contour data 22 in the data of a patient to be treated 2 and one of the respective diseased-site contour data 32 in the therapy plan database 3 have a mutually common region at parts of them, and FIG. 3B shows a situation where the diseased-site contour data 22 in the data of a patient to be treated 2 includes one of the respective diseased-site contour data 32 in the therapy plan database 3.

As shown in FIG. 3A and FIG. 3B, the diseased-site contour data 22 and each of the respective diseased-site contour data 32 are represented in coordinates. For example, assuming that one scale unit in the coordinates has an interval of 1 mm so that border lines are includes in a diseased site 10, in the situation of FIG. 3A, judging from the diseased-site contour data 22 in the data of the new patient to be treated 2, the number of coordinate points in the diseased site 10 is 56; judging from the diseased-site contour data 32 in the therapy plan database 3, the number of coordinate points in the diseased site 10 is 50; and the number of coordinate points in their common region (black circles in the figure) is 42.

In the situation of FIG. 3B, judging from the diseased-site contour data 22 in the data of the new patient to be treated 2, the number of coordinate points in the diseased site 10 is 56; judging from the diseased-site contour data 32 in the therapy plan database 3, the number of coordinate points in the diseased site 10 is 40; and the number of coordinate points in their common region (black circles in the figure) is 40.

In this manner, the number of coordinate points varies also in the Z-axis direction, so that, for example, the numbers of coordinate points as shown in Table 1 are provided.

TABLE 1
Number of Coordinate Points in Diseased Site

| | | Number of | Number of |
|---|---|---|---|
| | Number of | Coordinate Points | Coordinate Points |
| | Coordinate Points | in Diseased Site | in Common |
| Z-axis | in New Diseased Site | in Existing Case | Region |
| 1 | 0 (out of contour) | 0 (out of contour) | 0 (out of contour) |
| 2 | 0 (out of contour) | 2 | 0 (out of contour) |
| 3 | 15 | 20 | 14 |
| 4 | 70 | 75 | 65 |
| 5 | 75 | 80 | 73 |
| 6 | 60 | 60 | 50 |
| 7 | 10 | 10 | 7 |
| 8 | 2 | 0 (out of contour) | 0 (out of contour) |
| 9 | 3 | 0 (out of contour) | 0 (out of contour) |
| 10 | 0 (out of contour) | 0 (out of contour) | 0 (out of contour) |
| Total | 235 | 247 | 209 |

Here, the similarity (matching rate) is defined by a following formula (1).

(Similarity)=100×(Number of coordinate points at which coordinates in a new diseased site and coordinates in a diseased site in existing data are matched to each other)÷(Number of coordinate points of the coordinates in either one of the new diseased site and the diseased site in existing data, that are less than the coordinates in the other one) (1)

Further, the volume ratio (closeness in volume) is defined by a following formula (2).

(Volume Ratio)=100×(Number of coordinate points of coordinates in either one of a new diseased site and a diseased site in existing data, that are less than the coordinates in the other one)÷(Number of coordinate points of the coordinates in either one of the new diseased site and the diseased site in existing data, that are more than the coordinates in the other one) (2)

In the situation as shown in Table 1, there is provided:

(Similarity)=100×209÷235≈88.9(%)

(Volume Ratio)=100×235÷247≈95.1(%)

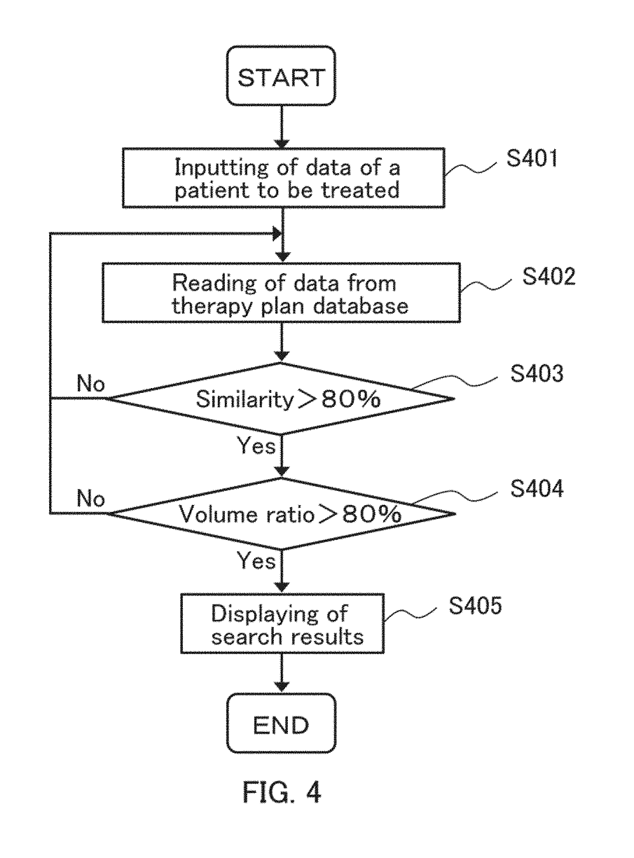

Next, operations of the therapy plan device 100 according to Embodiment 1 of the invention will be described based on FIG. 4. FIG. 4 is a block diagram for illustrating an existing therapy-plan data searching method by the therapy plan device 100 according to Embodiment 1 of the invention.

First of all, the CT image data 21 of the new patient to be treated is inputted as the data of a patient to be treated 2, so that the diseased-site contour data 22 and the body contour data 23 are read out, using image processing or the like, by the diseased-site contour data calculation unit 4 (Step S401). On this occasion, plural CT image data 21 of the cross-sections with respect to the body-axis direction are provided, said cross-sections being placed with specified intervals therebetween and corresponding to positions in the diseased site 10.

Subsequently, the CT image data 31 as existing therapy plan data, that is to be provided as a candidate, is read from the therapy plan database 3, so that its diseased-site contour data 32 and its body contour data 33 are read out, using image processing or the like, by the diseased-site contour data calculation unit 4 (Step S402). On this occasion, for example, the existing therapy plan data to be provided as the candidate is extracted from the data of an organ 11 corresponding to the diseased site 10 of the new patient to be treated.

Then, the diseased-site contour data calculation unit 4 compares the diseased-site contour data in the data of a patient to be treated 2 with the diseased-site contour data 32 in the therapy plan database 3 to thereby calculates the similarity, and the existing therapy-plan data search unit 5 determines whether or not the similarity is 80% or more (Step S403). If the calculated similarity is not 80% or more, other CT image data 31 as existing therapy plan data to be provided as a next candidate is read from the therapy plan database 3, so that its diseased-site contour data 32 and its body contour data 33 are read out, using image processing or the like, by the diseased-site contour data calculation unit 4 (Step S402).

When the calculated similarity is 80% or more, the diseased-site contour data calculation unit 4 compares the diseased-site contour data 22 in the data of a patient to be treated 2 with the diseased-site contour data 32 in the therapy plan database 3 to thereby calculates the volume ratio, and the existing therapy-plan data search unit 5 determines whether or not the volume ratio is 80% or more (Step S404). If the calculated volume ratio is not 80% or more, other CT image data 31 as existing therapy plan data to be provided as a next candidate is read from the therapy plan database 3, so that its diseased-site contour data 32 and its body contour data 33 are read out, using image processing or the like, by the diseased-site contour data calculation unit 4 (Step S402).

In this manner, the respective CT image data 31 each as existing therapy plan data to be provided as a candidate are all read sequentially; the treated cases with similarities of 80% or more and volume ratios of 80% or more, are searched from the therapy plan database 3 and extracted as similar cases for the new patient to be treated; and these search results are sorted and displayed in descending order of the similarities (Step S405).

Accordingly, it is possible to easily perform similarity searching from the existing therapy plan data in a short time without requiring advance preparation for that similarity searching with the existing therapy plan data, and a case (s) with a high similarity can be utilized as a similar case(s) for preparing a new therapy plan.

It is noted that, in Embodiment 1, the description has been made about a situation where the similarity search results are sorted and displayed in descending order of the similarities; however, this is not limitative. For example, with the inclusion of DVH (Dose Volume Histogram) information about a vital organ as therapy plan information in the therapy plan database 3, these results may instead be sorted in ascending order of radiation doses for the vital organ.

Further, with the inclusion of a therapy protocol as therapy plan information in the therapy plan database 3, these results may instead be sorted, for example, in descending order of information about local control rates or survival rates. Furthermore, with the inclusion of information about side effects as therapy plan information in the therapy plan database 3, these results may instead be sorted, for example, in ascending order of the side effects such as acute reactions, late-phase reactions or the like.

Further, when dose values at the intersections between the beam and the respective body contour data 33 are calculated from the respective body contour data 33 in the therapy plan database 3 and beam-related information in the respective therapy plan data 34 therein, these results may instead be sorted in ascending order of the dose values. Furthermore, with the inclusion of site-related information about a diseased site as therapy plan information in the therapy plan database 3, information of a vital organ or the like that is highly relevant to that site may be added as supplemental information.

As described above, in the therapy plan device 100 according to Embodiment 1 of the invention, the similarities and the volume ratios are calculated by the diseased-site contour data calculation unit 4 in such a manner that the diseased-site contour data 22 in the data of a patient to be treated 2 is compared with the respective diseased-site contour data 32 in the therapy plan database 3, using an origin point as a reference point; and then the similar case(s) is searched by the existing therapy-plan data search unit 5, based on the calculated similarities and the volume ratios;

said origin point being an intersection point between an X-axis and a Y-axis in a cross-section of the patient with respect to the body-axis direction, in which the X-axis corresponds to a line that passes a midpoint of a line segment connecting the front-end touch point A and the rear-end touch point B in a front-rear direction in the body contour data 12a, and that is perpendicular to the front-rear direction; and the Y-axis corresponds to a line in the front-rear direction that passes a midpoint of a line segment connecting the right-end touch point C and the left-end touch point D in a lateral direction in the body contour data 12a; and

said origin point also being an intersection point between a Z-axis in a longitudinal section along the Y-axis with respect to the body-axis direction and a sectional perpendicular to the X-axis and the Y-axis that passes the intersection point between the X-axis and the Y-axis, and the sectional plane passes a midpoint of a line segment connecting the touch point E and the touch point F at the both ends having a maximum width in the body-axis direction in the diseased-site contour data 10a.

Thus, it is possible to easily perform similarity searching from the existing therapy plan data in a short time without requiring advance preparation for that similarity searching with the existing therapy plan data, and a case(s) with a high similarity can be utilized as a similar case(s) for preparing a new therapy plan.

Further, since the relevant therapy plan information is included in the therapy plan database 3, it is possible not only to sort the searched similar cases in descending order of the similarities, but also to sort them in order based on the therapy plan information.

It should be noted that any appropriate modification/omission in the embodiments may be made in the present invention without departing from the scope of the invention.

### DESCRIPTION OF REFERENCE NUMERALS AND SIGNS

2: data of a patient to be treated, 3: therapy plan database, 4: diseased-site contour data calculation unit, 5: existing therapy-plan data search unit, 10: diseased site, 10a, 22, 32: diseased-site contour data, 12: patient, 12a, 23, 33: body contour data, 21, 31: CT image data, 34: therapy plan data, 35: dose data.

1-6. (canceled)

7. A therapy plan device, comprising:
data of a patient to be treated which includes first diseased-site contour data and first body contour data that are read from image data of the patient to be treated;
a therapy plan database which includes respective second diseased-site contour data and respective second body contour data that are read from respective image data of patients in treated cases;
a diseased-site contour data calculator for calculating similarities and volume ratios between the first diseased-site contour data and the respective second diseased-site contour data by comparing them using an origin point as a reference point,
said origin point being an intersection point between an X-axis and a Y-axis in a cross-section with respect to a body-axis direction, of each of the patient to be treated and the patients in the treated cases, in which the X-axis corresponds to a line that passes a midpoint of a line segment connecting a frontmost-end point and a rearmost-end point in a front-rear direction in each of the first body contour data and the respective second body contour data, and that is perpendicular to the front-rear direction; and the Y-axis corresponds to a line in the front-rear direction that passes a midpoint of a line segment connecting a rightmost-end point and a leftmost end point in a lateral direction in said each of the first body contour data and the respective second body contour data, and
said origin point also being an intersection point between a Z-axis in a longitudinal section along the Y-axis with respect to each said body-axis direction and a sectional plane, in which the Z-axis corresponds to a line perpendicular to the X-axis and the Y-axis that passes the intersection point between the X-axis and the Y-axis, and the sectional plane passes a midpoint of a line segment connecting both-end points having a maximum width in the body-axis direction in said each of the first diseased-site contour data and the respective second diseased-site contour data; and
an existing therapy-plan data searcher for searching a similar case(s), based on the similarities and the volume ratios.

8. The therapy plan device according to claim 7, wherein the similarities are each defined by a following formula (1).
(Similarity)=100×(Number of coordinate points at which internal coordinates of the first diseased-site contour data and internal coordinates of each of the respective second diseased-site contour data are matched to each other)÷(Number of coordinate points of the internal coordinates of either one of the first diseased-site contour data and said each of the respective second diseased-site contour data, that are less than the internal coordinates of the other one) (1)

9. The therapy plan device according to claim 7 wherein the volume ratios are each defined by a following formula (2).
(Volume Ratio)=100×(Number of coordinate points of the internal coordinates of either one of the first diseased-site contour data and each of the respective second diseased-site contour data, that are less than the internal coordinates of the other one)÷(Number of coordinate points of the internal coordinates of either one of the first diseased-site contour data and said each of the respective second diseased-site contour data, that are more than the internal coordinates of the other one) (2)

10. The therapy plan device according to claim 7, wherein the existing therapy-plan data searcher searches the similar cases each with the similarity and the volume ratio that are equal to or higher than their respective specified values.

11. The therapy plan device according to claim 8, wherein the existing therapy-plan data searcher searches the similar cases each with the similarity and the volume ratio that are equal to or higher than their respective specified values.

12. The therapy plan device according to claim 9, wherein the existing therapy-plan data searcher searches the similar cases each with the similarity and the volume ratio that are equal to or higher than their respective specified values.

13. The therapy plan device according to claim 7, wherein the similar cases are sorted and displayed in descending order of the similarities.

14. The therapy plan device according to claim 8, wherein the similar cases are sorted and displayed in descending order of the similarities.

15. The therapy plan device according to claim 9, wherein the similar cases are sorted and displayed in descending order of the similarities.

16. The therapy plan device according to claim 10, wherein the similar cases are sorted and displayed in descending order of the similarities.

17. The therapy plan device according to claim 11, wherein the similar cases are sorted and displayed in descending order of the similarities.

18. The therapy plan device according to claim 12, wherein the similar cases are sorted and displayed in descending order of the similarities.

19. The therapy plan device according to claim 7, wherein the therapy plan database includes therapy plan information and the similar cases are sorted and displayed in order based on the therapy plan information.

20. The therapy plan device according to claim 8, wherein the therapy plan database includes therapy plan information and the similar cases are sorted and displayed in order based on the therapy plan information.

21. The therapy plan device according to claim 9, wherein the therapy plan database includes therapy plan information and the similar cases are sorted and displayed in order based on the therapy plan information.

22. The therapy plan device according to claim 10, wherein the therapy plan database includes therapy plan information and the similar cases are sorted and displayed in order based on the therapy plan information.

23. The therapy plan device according to claim 11, wherein the therapy plan database includes therapy plan information and the similar cases are sorted and displayed in order based on the therapy plan information.

24. The therapy plan device according to claim 12, wherein the therapy plan database includes therapy plan information and the similar cases are sorted and displayed in order based on the therapy plan information.
